(19) United States
(12) Patent Application Publication
(10) Pub. No.: US 2018/0289242 A1
(43) Pub. Date: Oct. 11, 2018

(54) Endoscopy Steering Structure

(71) Applicant: Pioneer Medical Instrument Co., Ltd., New Taipei City (TW)

(72) Inventor: Chun-Yi Dai, New Taipei City (TW)

(73) Assignee: Pioneer Medical Instrument Co., Ltd.

(21) Appl. No.: 15/479,578

(22) Filed: Apr. 5, 2017

(51) Int. Cl. A61B 1/005; A61B 1/00; A61B 1/008; A61B 1/015; A61B 1/06; A61B 1/05

(52) U.S. Cl. CPC A61B 1/0057; A61B 1/00105; A61B 1/008; A61B 1/05; A61B 1/0055; A61B 1/015; A61B 1/0684; A61B 1/00096

(57) ABSTRACT

An endoscopy steering structure includes at least two pull strings and a steering module, and the pull string has a lower half of the pull string and an upper half of the pull string, and the steering module is connected to a lens module and a flexible detection tube, and the steering module has first and second hollow gears and a bending rib. The first hollow gear has first and second gullets, and tooth roots at the bottom of the first and second gullets abut the lower half of the pull string. The second hollow gear is next to the first hollow gear, and the second hollow gear has third and fourth gullets, and tooth roots of the third and fourth gullets abut the upper half of the pull string. The bending rib is integrally connected to the first and second hollow gear and bendable in both clockwise and anticlockwise directions.

### FIELD OF INVENTION

The present invention relates to the field of endoscopy, in particular to an endoscopy having a front end with a steerable structure.

### BACKGROUND OF INVENTION

### 1. Description of the Related Art

In general, a conventional endoscopy device is formed by connecting a photography module, a light emitting element, and a hollow tube. A built-in or external display screen is connected to display the image of a subject matter. For example, a medical endoscopy device is used to observe tissues inside a living body, or an industrial endoscopy device is used to observe the internal structure of a general object (such as a machine or transportation means), so that an operator can detect whether or not the subject matter is abnormal.

However, the conventional endoscopy steering mechanism has a plurality of hollow metal components, and each hollow metal component has a rivet hole corresponsive to a rivet, so that the metal structure can be steered. Therefore, the requirement for precision is very high, and a complicated manufacturing process is required to assemble a steering module with a pull string and a transmission line, and thus a high production cost and a complicated manufacturing procedure are incurred.

2. Summary of the Invention

In view of the aforementioned drawbacks of the prior art, the inventor of the present invention based on years of experience in the related industry to conduct extensive research and experiment, and finally developed an endoscopy steering structure in accordance with the present invention to overcome the drawbacks of the prior art.

Therefore, it is a primary objective of the present invention to provide an endoscopy steering structure, wherein the steering structure is made of an integral flexible material to provide the steering effect, and a gullet naturally formed between two tooth roots of a hollow gear is provided for installing two pull strings, and the two hollow gears are installed adjacent to each other, so that the upper half and the lower half of the pull string press the pull string from the tooth roots of the gears to achieve the effect of fixing the pull string, and a tooth root of a groove naturally formed between the teeth of the gear is provided for installing a plurality of pull strings and transmission lines. When the endoscopy steering structure is used for steering, the outer edges of the two hollow gears interfere with each other to achieve the effect of preventing the endoscopy steering structure from being steered freely or over-steered.

To achieve the aforementioned and other objectives, the present invention provides an endoscopy steering structure, comprising at least two pull strings, a steering module, a lens module and a flexible detection tube. The pull string has a lower half and an upper half. The steering module is connected to the lens module, and an end of the steering module is connected to a flexible detection tube, and the steering module has a first hollow gear, a second hollow gear 32 and a bending rib. The first hollow gear has a first tooth, a second tooth and a third tooth disposed adjacent to the left and right sides of the first tooth respectively, a first gullet disposed between the first tooth and the second tooth, a second gullet disposed between the first tooth and the third tooth, tooth roots at the bottom of the first gullet and the second gullet abut the lower half of the pull string. The second hollow gear is disposed adjacent to the first hollow gear, and the second hollow gear has a fourth tooth, a fifth tooth and a sixth tooth disposed adjacent to the left and right sides of the fourth tooth respectively, a third gullet disposed between the fourth tooth and the fifth tooth, a fourth gullet disposed between the fourth tooth and the sixth tooth, and tooth roots at the bottom of the third gullet and the fourth gullet abut the upper half of the pull string. The bending rib has a front end integrally connected to the first hollow gear and a rear end integrally connected the second hollow gear, and the cross-section of the bending rib has a short side and a long side, and the bending rib is bendable from the long side and in both clockwise and anticlockwise directions.

### BRIEF DESCRIPTION OF THE DRAWINGS

FIG. 1 is a perspective view of an endoscopy steering structure in accordance with the present invention;

FIG. 2 is a perspective view of an endoscopy steering structure and a lens module in accordance with the present invention;

FIG. 3 is a perspective view of an endoscopy steering structure in accordance with the present invention after a glue is filled into a lens module;

FIG. 4 is a schematic view of an endoscopy steering structure in accordance with the present invention after the endoscopy steering structure is steered partially;

FIG. 5 is a schematic view of an endoscopy steering structure in accordance with the present invention after the endoscopy steering structure is steered clockwise; and

FIG. 6 is a schematic view of an endoscopy steering structure in accordance with the present invention after the endoscopy steering structure is steered anticlockwise.

### DESCRIPTION OF THE PREFERRED EMBODIMENTS

The above and other objects, features and advantages of this disclosure will become apparent from the following detailed description taken with the accompanying drawings.

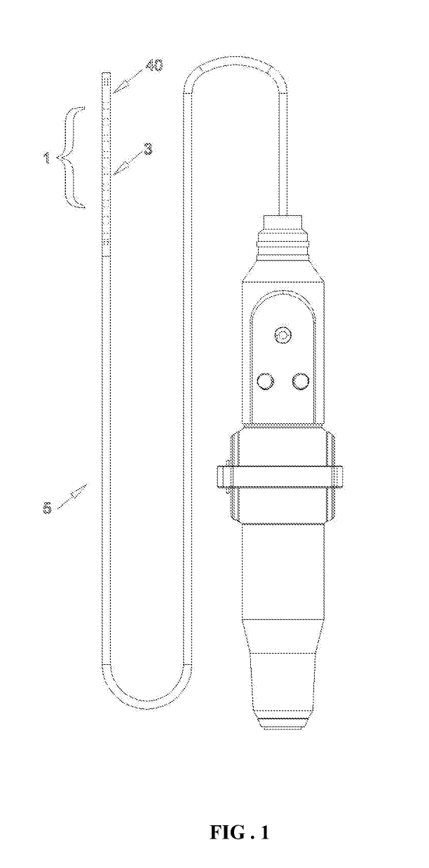

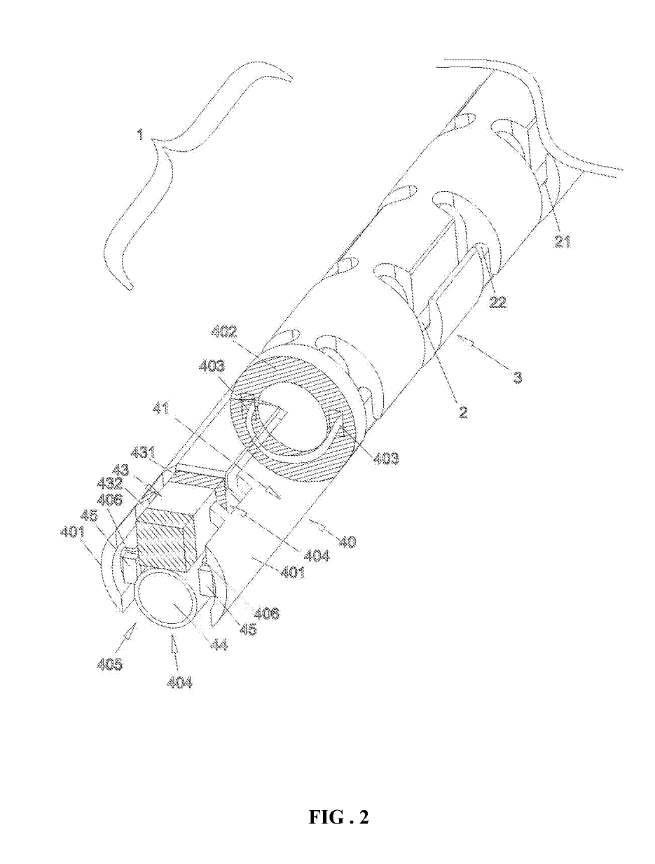

With reference to FIGS. 1 and 2 for a perspective view of an endoscopy of an endoscopy steering structure and a perspective view of the steering structure with a lens module in accordance with the present invention respectively, the endoscopy steering structure 1 comprises at least two pull strings 2, a steering module 3, a lens module 40, and a flexible detection tube 5.

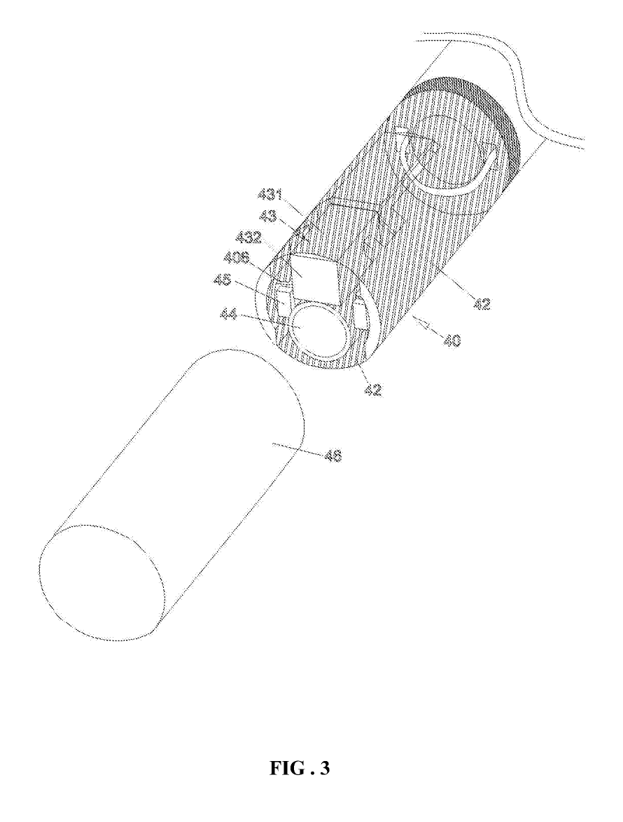

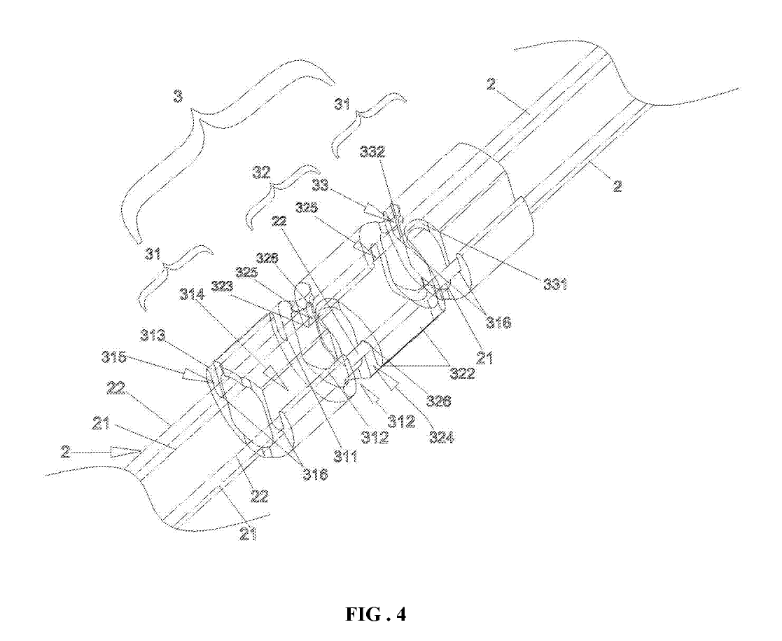

With reference to FIGS. 2 to 4 for a perspective view of an endoscopy steering structure and a lens module, a schematic view of a lens module after being filled with a glue, and a schematic view of a partial steering structure in accordance with the present invention, each pull string 2 has a lower half 21 and an upper half 22. The steering module 3 is integrally connected to a lens module 4 for preventing the steering module 3 from being broken, cracked, or elastically fatigue at the position where the steering module 3 and the lens module 4 are bent repeatedly. An end of the steering module is connected to the flexible detection tube 5, wherein the steering module 3 is made of a polymer such as ABS, PP, PE, POM, nylon, silicone or rubber, and the steering module has a first hollow gear 31, a second hollow gear 32, and a bending rib 33.

The first hollow gear 31 has a first tooth 311, a second tooth 312 and a third tooth 313 disposed adjacent to the left and right sides of the first tooth 311 respectively, a first gullet 314 disposed between the first tooth 311 and the second tooth 312, a second gullet 315 disposed between the first tooth 311 and the third tooth 313, and a tooth root 316 at the bottom of the first gullet 314 and the second gullet 315 abuts the lower half of the pull string 21. The second hollow gear 32 is disposed adjacent to the first hollow gear 31, and the second hollow gear 32 has a fourth tooth 321, a fifth tooth 322 and a sixth tooth 323 disposed adjacent to the left and right sides of the fourth tooth 321 respectively, a third gullet 324 disposed between the fourth tooth 321 and fifth tooth 322, a fourth gullet 325 disposed between the fourth tooth 321 and the sixth tooth 323, and a tooth root 326 at the bottom of the third gullet 324 and the fourth gullet 325 abuts the upper half 22 of the pull string.

As described above, the hollow gears 31, 32 and the gullets 314, 315, 324, 325 naturally formed between two tooth roots 316, 326 of the hollow gears 31, 32 and provided for installing two pull strings 2, and then the two hollow gears 31, 32 are installed adjacent to each other, so that the two tooth roots 316, 326 of the hollow gears 31, 32 abut inwardly at the pull string, so that the upper and lower halves 21, 22 achieve the effects of positioning the pull string 2 and steering the endoscopy easily. In addition, the tooth roots 316, 326 of the grooves naturally formed between the teeth of the hollow gears 31, 32 are provided for installing a plurality of pull strings 2 and transmission lines.

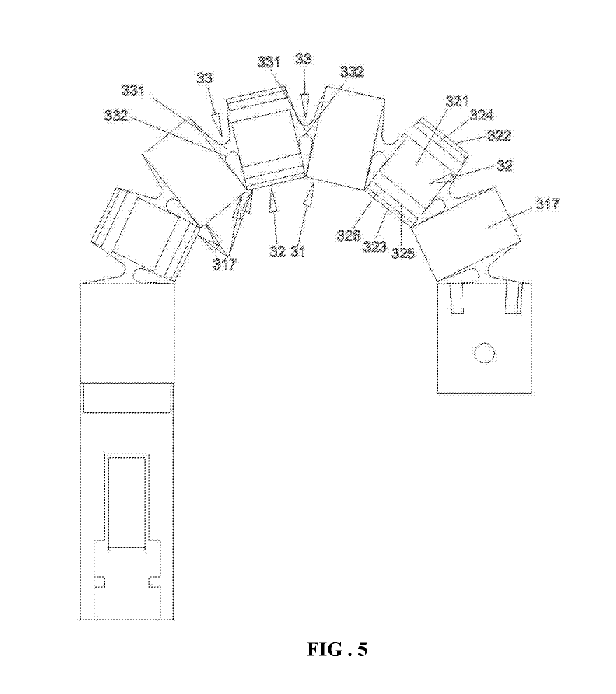

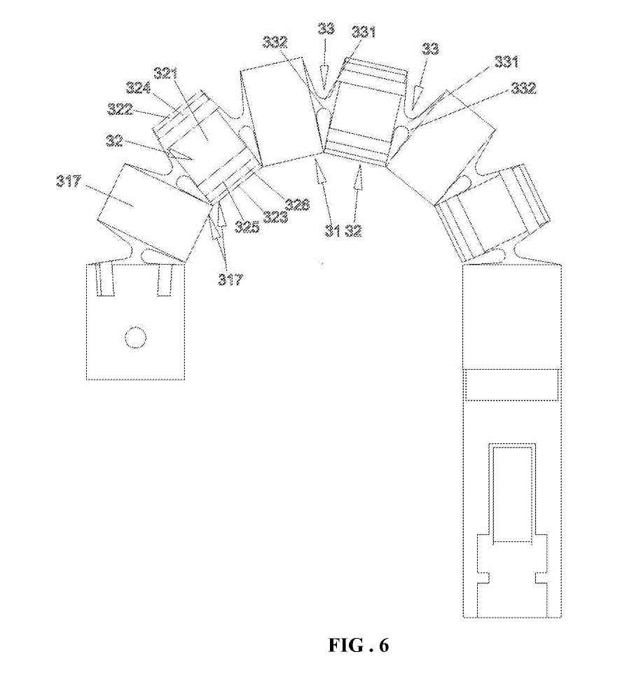

It is noteworthy that the tooth root at the bottom of each gullet is lower than the center position of the hollow gear, so that the gear and its adjacent gears have the same structure (wherein the adjacent gears just need to be steered, so that the first gullet 314 and the second gullet 315 of the first hollow gear 31 are opposite to the third gullet 324 and the fourth gullet 325 of the second hollow gear 3) to achieve the effects of abutting the lower half 21 of the pull string 2 and the upper half 22 of the pull string 2 or the transmission line, so as to position the pull string 2 and achieve a steering effect easily. Each tooth root of the aforementioned gears is lower than the center position of the hollow gear, and spaced from a general gear, so that the same gears 31, 32 can be installed continuously to form a steering module 3. The bending rib 33 has a front end integrally connected to the first hollow gear 31 and a rear end integrally connected to the second hollow gear 32, and the cross-section of the bending rib 33 has a short side 331 and a long side 332, and the bending rib 33 is bendable from the long side 332 and in both clockwise and anticlockwise directions as shown in FIGS. 5 and 6.

The second tooth 312 is a circular arc body 317 bending in an opposite direction away from the first tooth 311 and clockwise to the outer edge of the third tooth 313, wherein the fifth tooth 322 is a circular arc body 317 bending in an opposite direction away from the fourth tooth 321 and anticlockwise to the outer edge of the sixth tooth 323. When one of the pull strings 2 is pulled, the bending rib 33 bends clockwise or anticlockwise, and the circular arc body 317 of the outer edge of the first hollow gear 31 and the circular arc body 317 of the outer edge of the second hollow gear 32 interfere with each other to limit the bending angle of the bending rib 33. As described above, the interference between the outer edges of the two adjacent hollow gears 31, 32 is used to achieve the effects of preventing the endoscopy steering structure from being steered freely and preventing the steering angle from being too large.

The lens module 40 has a double crescent rib 401 extended axially from the outer edge of a circular hollow base 402, and the crescent rib 401 and the circular hollow base 402 define a hollow accommodating space 41, and the outer edge of the double crescent rib 401 is equal to 1/1.5(2πr)˜1/6(2πr) of the perimeter of the outer edge of the circular hollow base 402, wherein the circular hollow base 402 further has two pull string passing holes 403 for passing the pull strings 2 and fixing the pull strings 2 by a filling glue 42. The hollow accommodating space 41 is provided for accommodating an imaging module 43, and the imaging module 43 has an image sensor 431, a lens 432 and an LED 44, wherein the LED 44 is installed onto an L-shaped LED positioning seat 406.

The lens module 40 further has a hollow passage 44 disposed in the hollow position of the lens module 40, extended from the hollow position of the steering module 3 to the hollow position of the flexible detection tube 5, and provided for transmitting a gas/liquid or passing components.

Wherein, a placement opening 404 is formed between the crescent ribs 401, and an opening 405 is formed between the front ends of the crescent ribs 401 for receiving images from the front. In addition, the placement opening 404 formed between two crescent ribs 401 increases the hollow accommodating space 41 slightly. When the components including the image sensor 431, lens 432 and LED are installed in the hollow accommodating space 41 and occupy too much external periphery, the space of the placement opening 404 accommodates some of the volume and external periphery of the image sensor 431, lens 432 and LED 45, and then the filling glue 42 is used to fix and fill in the remaining space of the hollow accommodating space 41, so as to achieve the cooling effect by the thermal conduction of the filling glue 42.

The filling glue 42 is filled into the hollow accommodating space 41 from the opening 405 formed between the front ends of the two crescent ribs 401, and the hollow accommodating space 41 is defined by the crescent rib 401 and the circular hollow base 402. The circular arc sleeve 46 is sheathed on the lens module 40 from the opening 405 formed b the front ends of the two crescent ribs 401, so that the filling glue 42 is filled into hollow accommodating space 41 and up to the placement opening 404, and the placement opening 404 disposed between the two crescent ribs 401 may increase or decrease the external periphery of the lens module 40 flexibly to accommodate the components in the hollow accommodating space 41, and then the circular arc sleeve 46 is removed to achieve the effect of designing the lens module 40 with a housing of any size.

While the invention has been described by means of specific embodiments, numerous modifications and variations could be made thereto by those skilled in the art without departing from the scope and spirit of the invention set forth in the claims.

What is claimed is:

1. An endoscopy steering structure, comprising:
at least two pull strings, each having a lower half and an upper half; and
a steering module, coupled to a lens module, and having an end coupled to a flexible detection tube, and the steering module further comprising:
a first hollow gear, having a first tooth, a second tooth and a third tooth disposed adjacent to the left and right sides of the first tooth respectively, a first gullet disposed between the first tooth and the second tooth, a second gullet disposed between the first tooth and the third tooth, a tooth root at the bottom of the first gullet and the second gullet abutting the lower half of the pull string;
a second hollow gear, disposed adjacent to the first hollow gear, and having a fourth tooth, a fifth tooth and a sixth tooth disposed adjacent to the left and right sides of the fourth tooth respectively, a third gullet disposed between the fourth tooth and the fifth tooth, a fourth gullet disposed between the fourth tooth and the sixth tooth, and a tooth root at the bottom of the third gullet and the fourth gullet abutting the upper half of the pull string; and
a bending rib, having a front end integrally coupled to the first hollow gear, and a rear end integrally coupled to the second hollow gear, and the bending rib having a cross-section which is a short side and a long side, and the bending rib being bendable from the long side and in both clockwise and anticlockwise direction.

2. The endoscopy steering structure of claim 1, wherein the second tooth is a circular arc body bending in a direction opposite to the first tooth and clockwise to an outer edge of the third tooth, and the fifth tooth is a circular arc body bending in a direction opposite to the fourth tooth and anticlockwise to an outer edge of the sixth tooth, so that if one of the pull strings is pulled, the bending rib will bend clockwise or anticlockwise, and the circular arc body of the first hollow gear and the circular arc body of the second hollow gear will interfere with each other to limit of the bending rib bending angle.

3. The endoscopy steering structure of claim 1, wherein the lens module further has a hollow accommodating space and a hollow passage, and the hollow accommodating space is provided for accommodating an imaging module, and the imaging module has an image sensor, a lens, and an LED, and the LED is installed to an L-shaped LED positioning seat, and the hollow passage is extended from the hollow of the steering module to the hollow of the flexible detection tube and provided for transporting a gas/liquid or passing a component.

4. The endoscopy steering structure of claim 1, wherein the lens module has a double crescent rib, and the double crescent rib is extended axially from the outer edge of a circular hollow base, and the crescent rib and the circular hollow base define a hollow accommodating space, and the outer edge of the double crescent rib falls within a range of 1/1.5(2πr)˜1/6(2πr) of the perimeter of the outer edge of the circular hollow base.

5. The endoscopy steering structure of claim 4, further comprising a placement opening formed between the crescent ribs, and an opening formed between the fronts of the crescent ribs.

6. The endoscopy steering structure of claim 1, further comprising a filling glue filled from an opening formed between the fronts of the crescent ribs into a hollow accommodating space, and the hollow accommodating space being defined by the crescent rib and the circular hollow base.

7. The endoscopy steering structure of claim 6, wherein the circular arc sleeve is sheathed on the lens module from the opening at the front end of the two crescent ribs, so that the filling glue is filled into the hollow accommodating space and filled up to a placement opening, and the placement opening is disposed between the two crescent ribs.
